(19) United States
(12) Patent Application Publication
(10) Pub. No.: US 2020/0164079 A1
(43) Pub. Date: May 28, 2020

(54) ANTI-BACTERIAL SUBSTANCE AND COMPOSITION THEREOF, MEDICAL AND NON-MEDICAL USES USING THE SAME, AND PRODUCTS COMPRISING SAID SUBSTANCE AND COMPOSITIONS

(71) Applicant: PVAC MEDICAL TECHNOLOGIES LTD, NICOSIA (CY)

(72) Inventors: Thomas ENGSTRAND, UPPSALA (SE); David BERGLUND, UPPSALA (SE); Carl PÅLSSON, UPPSALA (SE); Folke KNUTSON, UPPSALA (SE)

(21) Appl. No.: 16/632,096

(22) PCT Filed: Jul. 17, 2018
(86) PCT No.: PCT/EP2018/069356

§ 371 Date: Jan. 17, 2020

(30) Foreign Application Priority Data
Jul. 18, 2017 (SE) 1750946-4

(51) Int. Cl. A61K 47/58; A61L 29/16; A61L 29/12

(52) U.S. Cl. CPC A61K 47/58; C08L 29/04; A61L 29/126; A61L 29/16

(57) ABSTRACT

An active principle for use in prevention or treatment of bacteria-induced infection and disease, wherein the active principle includes a carrier which exhibits a plurality of a scavenger structure which is capable of interacting with bacteria and reducing its growth and/or adhesion to a surface, the scavenger structure including a nucleophilic center complying with the formula X¹(—R″—)(—R′)ₘHₙ (formula I) where a) X¹ is a single-bonded heteroatom selected amongst N, O and S and exhibits a free electron pair, b) m is 0 or 1 and n is 1 or 2, c) —R″— is a bivalent organic group providing attachment to the carrier via one of its free valences and to X¹ at the other free valence, and d) R′— is a monovalent organic group attached to the X¹ via its free valence.

### TECHNICAL FIELD

The present invention relates to an active principle or a composition thereof for prevention and treatment of bacteria and bacterial infections for medical and non-medical uses. The present invention further relates to products comprising said active principle or a composition thereof for medical as well as non-medical applications.

### BACKGROUND TECHNOLOGY

Exposure to bacteria may cause deleterious infections in humans and animals. Antibiotics may either kill or inhibit growth of bacteria. Alternatively, the prevention of bacterial adhesion to surfaces, cells and tissues by drugs that interfere with bacterial cell-surface components have been proposed as a method to lower bacterial virulence. The extensive overuse of antibiotics to prevent or treat infections have caused antibiotic resistance in bacteria, which today represent one of the biggest threats to human health.

Polymers or other macromolecules have previously been utilized in conjugation with antimicrobial agents as excipients. Macromolecules may be functionalized with active groups that interfere with bacterial components and may constitute a compound for the treatment or prevention of bacterial contaminations in vitro. Polymers carrying antimicrobial agents via an alkyl or acetyl linker are described. These antimicrobial polymers are typically used as surface coatings in containers, filters, packaging materials, etc, for the prevention and treatment of microorganism contaminations and bacterial growth for water treatment, in food applications and antimicrobial treatment of different implant materials. However, a general concern is potential solubility in water since several of these polymer-attached antimicrobial agents are toxic. The in vivo-use of such agents for the prevention or treatment of infections is thus restricted.

### SUMMARY OF THE INVENTION

The present inventors have recognised that immobilization of relevant scavenger functionalities to a macromolecular carrier would improve the antibacterial effects of the corresponding low molecular weight scavenger molecule. Unexpectedly it has been found that the optimal functionalities have been found amongst reactive groups that normally are used for cross-linking macromolecular carriers and/or for the synthesis of carrier-bound therapeutic active entities (WO 2009108100 (belonging to the inventors) and references cited therein). In WO 2009108100 these substances were used to treat inflammatory conditions caused by various antigens. The inventors later discovered that the same substances had an antibacterial effect in that they affected the adhesion of the bacteria to surfaces and/or the growth of the bacteria. Thus, the substances may thus also be used to prevent and treat bacterial-induced infection.

In the present specification our principle is demonstrated by the synthesis of functionalized carriers or active principles which are capable of binding to bacteria and reducing its capacity to proliferate and/or binding to a surface. In the experimental part, this kind of construct is shown to significantly reduce the growth of 12 common bacteria in vitro (Example 1).

Without wishing to be bound by any theory, the binding of bacteria to surfaces, corporal or other, is believed to be perturbed by the antibacterial substances according to the invention. The substances may also interfere with the actual proliferation process of bacteria.

From a bacterial resistance perspective, the development of an agent with different antimicrobial mechanistic effect would be beneficial because it may constitute a sole alternative or adjuvant to conventional antimicrobial agents. Also, as compared to smaller agents, the molecular size of the substances according to the present invention effect pharmacokinetic and distribution profiles in a way that may be advantageous for certain indications where a prolonged and directed antimicrobial effect is needed. Other potential benefits include excellent in vivo tolerance and low manufacturing costs.

### FIRST MAIN ASPECT (ACTIVE PRINCIPLE) AND EMBODIMENTS THEREOF

An active principle for use in the prevention and treatment of bacteria-induced infection and disease, wherein the active principle comprises a carrier which exhibits a plurality of a scavenger structure, said scavenger structure comprising a nucleophilic centre complying with the formula

X¹(—R″—)(—R′)ₘHₙ (I)

wherein
a) X¹ is a single-bonded heteroatom selected amongst N, O and S and exhibits a free electron pair,
b) m is 0 or 1 and n is 1 or 2 with the sum of m plus n being 2 for X¹=N and 1 for X¹=S and O,
c) —R″— is a bivalent organic group providing attachment to the carrier via one of its free valences and direct attachment to the heteroatom X¹at the other one of its free valences, and
d) R′— is a monovalent organic group directly attached to the heteroatom X¹ via its free valence.

According to an embodiment of the active principle, either one or both of the organic groups R′— and —R″—, with preference for —R″—, comprise a structure of the formula:

—CH₂(X⁴)_{o′}(C═X³)^{n′}(X²)_{m′}— (II)

wherein
a) each of m′, n′ and o′ is 0 or 1, with preference for m′ being 1 with further preference for either one or both of n′ and o′ also being 1,
b) each of X² , X³, and X⁴, is selected amongst NH and a heteroatom S or O, with preference for either one or both of X² and X⁴ being selected amongst NH and O with further preference for X³ being selected amongst NH, O and S,
c) the left free valence provides binding to a monovalent alkyl group R*— or to the carrier via at least a bivalent alkylene group —R**—, each of which two groups comprises the methylene group —CH₂— shown in formula II, and
d) the right free valence binds directly to the first heteroatom X¹.

According to another embodiment of the active principle, the nucleophilic center is part of a group selected amongst
a) amino groups, preferably primary or secondary amino groups,
b) hydrazide groups such as —NH—NH₂, e.g. as part of a —CONHNH₂ group, a semicarbazide group such as —NHCONHNH₂, a carbazate group such as —OCONHNH₂, a thiosemicarbazide group such as —NHCSNHNH₂, a thiocarbazate group such as —OCSNHNH₂,
c) aminooxy groups, such as —ONH₂, etc, and
d) thiol groups, e.g. —SH.

According to a still further embodiment of the active principle, the carrier is a macromolecular carrier and/or is water-soluble or water-insoluble and preferably exhibits polymer structure. The carrier may be water-insoluble and define a support and/or the active principle is attached to a water-insoluble support.

According to another embodiment, the active principle has a scavenger structure capable of undergoing an addition reaction with a carbonyl group of an aldehyde group and/or with a reactive carbon-carbon multiple bond to which is directly attached a carbonyl group, such as an aldehyde group.

In one specific embodiment, the active principle is carbazate-functionalized polyvinyl alcohol.

In a further embodiment, the active principle is in liquid or solid form.

The active principle defined above is used in prevention or treatment of bacteria-induced infection and disease in connection with mouth, dental or throat infections (such as included in a mouthwash or gargling solution), systemic infection, alone or as an adjuvant treatment to antibiotics (for sepsis e.g. parenteral injection or infusion solution; for an antibacterial effect and improving the biota in the gastrointestinal tract e.g. peroral administration of drinkable solutions or tablets or capsules), infections in skin sores or ulcers (such as a sanitizing solution, skin cream or ointment, wound dressings, etc), eye infections (such as included in eye drops), ear infections (such as included in ear drops), cystitis in individuals with catheter (such as included in an irrigation solution), venereal diseases in individuals (such as included in local topical application of a skin cream or ointment in genital regions), bacterial growth in bags containing blood or other blood components, including plasma and thrombocytes (direct injected in the bag or as part of a surface coating on the inner surface of the bag, respiratory infections (such as included in an inhalation spray or aerosol), infections deep in body cavities or abscesses (such as by direct injection into such areas), central nervous infections, such as meningitis (such as by intrathecal injection), or prevention of bacterial infections in immunosuppressed patients (such as an additive to bone marrow transplants).

### SECOND MAIN ASPECT (COMPOSITION) AND EMBODIMENTS THEREOF

According to a second aspect, there is provided a pharmaceutical composition for use in prevention and treatment of bacterial-related infection and disease, comprising the active principle mentioned above and a buffer, saline solution, and/or other pharmaceutically suitable adjuvants. The pharmaceutical compositions may for example be combined with gels, freeze-dried collagen powder, hemostatic gauze, nanoparticles, or micelle-forming lipids depending on the administration routes.

The pharmaceutical composition may be used for the same type of bacterial-related infection and disease as mentioned for the active principle above.

According to one embodiment, the composition is in liquid or solid form.

In a further embodiment, said active principle is present in the pharmaceutical composition is present at a concentration of 5 μg/ml or more, or 10 μg/ml or more, or 20 μg/ml or more, or 100 μg/ml or more, or 200 μg/ml or more, or 500 μg/ml or more, or 20 mg/ml or less, but preferably 10 mg/ml or less, or 5 mg/ml or less, or 1 mg/ml or less.

### THIRD MAIN ASPECT (METHOD)

This aspect relates to the use of the above-mentioned active principle or pharmaceutical composition (defined above under the first and second main aspects) for prevention or treatment of bacteria-induced infection and disease as listed above in connection with the description of the active principle and the pharmaceutical composition.

In the above-mentioned methods for prevention and treatment of bacteria-induced infections and disease, the active principle or the pharmaceutical composition mentioned above would be administered to a patient in need thereof. The administration ways or routes may vary according to the specific bacterial infection or medical situation and are part of the knowledge of a medical practioner.

The administration may be done locally or systemically or in combination. Some are given above as illustrative examples of such administration ways or routes.

### FURTHER MAIN ASPECTS (PRODUCTS) AND EMBODIMENTS THEREOF

A non-medical antibacterial composition comprising the active principle as defined above and a suitable carrier and/or an adjuvant. In one embodiment said composition is used for sanitizing to eliminate or reduce bacteria on at least part of inner and/or outer surfaces of an object.

A mouthwash or gargling solution for reducing the bacterial content in the oral cavity to improve the smell of the breath, comprising the active principle as defined above and a suitable carrier and/or an adjuvant

An antibacterial sanitizing solution comprising the active principle as defined above and a suitable carrier and/or adjuvant for pre- or perioperative sanitizing of medical implants, including but not limited to catheters and stents, at the time of surgery.

A surface coating comprising the active principle or the non-medical antibacterial composition defined above.

An object having an inner and an outer surface, wherein at least part of the inner surface and/or outer surface exhibit the antibacterial surface coating as defined above. In further embodiments, the object is a blood bag, a stent, a catheter, or a medical device or implant.

In a further embodiments, said active principle is present in the above mentioned non-medical compositions or solutions, or coatings, at a concentration of 5 μg/ml or more, or 10 μg/ml or more, or 20 μg/ml or more, or 100 μg/ml or more, or 200 μg/ml or more, or 500 μg/ml or more, or 20 mg/ml or less, but preferably 10 mg/ml or less, or 5 mg/ml or less, or 1 mg/ml or less.

### BRIEF DESCRIPTION OF THE DRAWINGS

FIG. 1 shows a bar diagram of antibacterial treatment of twelve different bacterial strains using PVAC (polyvinyl alcohol carbazide). Optical density (OD) was determined after 2.5 hours of incubation. All bacterial species demonstrated a minimal growth at 2.5 h compared to the Oh baseline (dotted line). The figure displays a representative example, wherein 1.25 mg/ml of PVAC was used in the incubation of 10³-10⁵ bacteria. FIG. 2 is a photo of the anti-bacterial effect of PVAC, demonstrating a substantial reduction in the number of colony-forming units (CFUs) of E. coli bacteria. To the left, the non-PVAC-containing control displays a saturated number of CFUs, so great that they are unable to be counted in detail (>1000). On the right, bacteria treated with PVAC are unable to grow freely and produce only 75 CFUs.

### DETAILED DESCRIPTION OF THE INVENTION

The present invention involves:
i) providing an active principle or pharmaceutical composition thereof comprising a carrier which exhibits a plurality of a scavenger structure, and
ii) contacting AP with said bacteria or bacterial infection a) within said individual, or b) separate from said individual.

Alternative (a) means that the composition containing AP is administered to said individual.

The individual is typical an animal, such as a vertebrate, with particular emphasis of a mammal such as a human being. In the case the method is a therapeutic treatment the individual is typically a patient.

Step (ii.a) means that the contacting of bacteria or bacterial infection is taken place in vivo, i.e. within the body, on the skin, etc of the individual suffering from or being at risk for bacterial infections.

### The Composition

The composition may contain one or more formulations where at least one of them comprises AP or reagents necessary for the formation in vivo of AP (e.g. as described in WO 2009108100 for compositions used for formation in vivo of extracellular matrices).

### The Active Principle (=AP)

AP comprises a carrier exhibiting the plurality of the scavenger structure. Every scavenger structure is firmly attached to the carrier, for instance covalently. AP as well as the carrier as such may be soluble or insoluble in aqueous liquids such as water, body fluids, such as blood, serum, plasma, urine, lymph, lachrymal fluid, intestinal juice, gastric juice, saliva, synovial fluid, etc.

AP may be fixed to a water-insoluble support that may be of various physical and/or geometric appearances depending on the intended use. This is described further below.

Either one or both of the carrier and the support should be inert in the sense that they should not participate as competing reactants in the reaction between the scavenger structure and the bacteria. Both of them should have an acceptable biocompatibility causing low or no host defence reactions including low or no inflammation.

### The Scavenger Structure

The scavenger structure when present on the carrier mitigates and/or neutralizes the growth and/or adhesion of the bacteria.

The scavenger structure comprises a first nucleophilic centre which preferably is capable of participating in an addition reaction with the carbonyl group (C═O) of an aldehyde group, -and/or with a C,C-multiple bond to which one or more electron-withdrawing substituents preferably are directly attached.

The nucleophilic centre (first centre) of the scavenger structure preferably comprises a single-bonded first heteroatom N, O or S (=X¹) which exhibits
a) a free electron pair,
b) one or two hydrogens, and
c) one or two organic groups R′— (monovalent) and —R″— (divalent) directly bound to the heteroatom.

The preferred heteroatoms are N and S. S is preferably combined with the presence of a second nucleophilic centre, such as a primary or secondary amino, in the same scavenger structure as discussed below. The bivalent organic group —R″— provides binding to the carrier via one of its free valencies. The other free valency of —R″— as well as the free valency of the other organic group R′— are directly attached to the heteroatom X¹.

Generically a nucleophilic centre has the formula:

X¹(—R″—)(—R′)ₘHₙ (I)

where X¹, R′— and —R″— are as defined in the preceding paragraph and m is 0 or 1 and n is 1 or 2 with the sum of m plus n being 2 for X¹=N and 1 for X¹=S and O.

A single-bonded atom means that the atom is directly bound to other atoms only by single bonds. A multiple-bonded atom means that the atom is directly bound to another atom by a triple or a double bond. The atoms referred to are primarily N, O, S and carbon.

The preferred nucleophilic centres are typically uncharged when interacting with the bacteria. For a nucleophilic centre which is an uncharged base or acid form of an acid-base pair ≥5%, such as ≥25% or ≥50 or ≥75%, of the total concentration of the acid-base pair should be in uncharged form.

When the heteroatom X¹ is N, the ability to react with an aldehyde group will include that the adduct formed is capable of undergoing spontaneous elimination of water (H₂O) to the formation of an imine structure (—CH═NR″—, m=0 and n=2) and/or an enamine structure (—CH═CHNHR″—, m=0 and n=2) or —CH═CHNR′R″—, m=1 and n=1) with both alternatives requiring a hydrogen (α-hydrogen) on a sp³-hydridised α-carbon of the aldehyde group —CHO). When the heteroatom X¹ is S or O, m=0 and n=1 which means that the structure obtained upon elimination of H₂O is thioeno late or enolate (—CH═CHX′R′) (provided there is an α-hydrogen of the aldehyde group —CHO). These elimination reactions typically mean formation of a more stable product and/or a product that may react further to a further stabilized “end”-product. The selection of scavenger structures containing groups permitting subsequent reactions which end up in stabilized end products will support irreversibility of the initial addition reaction, and are as a rule preferred.

The reaction of the first nucleophilic centre and a reactive C,C-multiple bond on a acetaldehyde will result in a primary adduct which comprises the structure >CH—CHX¹— (for C,C— double bonds) and if the multiple bond is α,β to an aldehyde group there can be formed different tautomeric adducts. e.g. —CHX¹—CHαCHOH (enol) and —CHX¹—CH₂—CH═O (keto) which will enable another nucleophilic centre of the same or another scavenger structure to react with the bacteria.

Either one or both of the organic groups R′— and —R″— comprise a structure of the formula

—CH₂(X⁴)_{o′}(C═X³)_{n′}(X²)_{m′}— (II)

where
a) each of m′, n′ and o′ is 0 or 1, with preference for m′ being 1 with further preference for either one or both of n′ and o′ also being 1,
b) each of X², X³, and X⁴, is selected amongst NH and a heteroatom S or O, with preference for either one or both of X² and X⁴ being selected amongst NH and O with further preference for X³ being selected amongst NH, O and S,
c) the left free valence provides binding to a monovalent alkyl group R*— or to the carrier via at least a bivalent alkylene group —R**—, each of which comprises the methylene group —CH₂— shown of formula II,
d) the right free valency binds directly to the first heteroatom X′.

The substructure C═X³ (═B) includes also other ester- and amide-forming substructures which derive from acid functions and form an ester function when X² and/or X⁴ are oxygen and/or an amide function when X² and/or X⁴ are NH, e.g. sulphonamide (B is S(═O)₂) or phosphone amide (B is P═O(NH₂) or P═O(OH), n′=1).

Either one or both of the monovalent alkyl group R*— and the bivalent alkylene —R**— may be straight, branched or cyclic and possibly contain one or more structures selected amongst ethers (—O—, —S—), hydroxy (—OH), mercapto (—SH) and amino (—NH—, —NH₂). Each free valences represent binding to sp³-hybridised carbon (=alkyl carbon). Either one or both of these alkyl groups are preferably a lower alkyl which in this context means that they comprise one, two, three, four, five up to ten sp^{a}-hybridised carbons typically with at most one heteroatom O, N and S bound to one and the same carbon. The groups are typically inert in the sense that they are not participating in the reaction which interferes with the bacteria. The hydrogens given in formula (I) and/or its substructures may be replaced with an alkyl group selected amongst the same alkyl groups as discussed for R*—.

It is preferred that the bivalent group —R″— which attaches the first nucleophilic centre to the carrier comprises a substructure complying with formula I and/or II.

The structural elements (substructures) discussed in the preceding paragraphs will support delocalisation of electrons and therefore further support irreversibility of the initial addition reaction.

Preferred scavenger structures thus have a nucleophilic centre which contain the first heteroatom X¹ together with a structure complying with formula II and are selected amongst:
a) amino groups preferably primary or secondary amino groups
b) hydrazide groups such as —NH—NH₂, e.g. as part of a —CONHNH₂ group, a semicarbazide group such as —NHCONHNH₂, a carbazate group such as —OCONHNH₂, a thiosemicarbazide group such as —NHCSNHNH₂, a thiocarbazate group such as —OCSNHNH₂ (formation of hydrazone, semicarbazone, thiocarbazone linkages/groups, etc when undergoing addition/elimination reactions with an aldehyde group)
c) aminooxy groups, such as —ONH₂, etc (formation oxime linkages/groups, etc when undergoing addition/elimination reactions with an aldehyde group),
d) a thiol group e.g. —SH (Michael addition products are formed when the thiol group reacts with a C,C-double bond. The product may undergo keto-enol tautomerisation when the double bond is α,β to a keto- or aldehyde-carbonyl, see above).

The free valence indicated in each of the groups given in the preceding paragraph preferably attaches the nucleophilic centre to the carrier via a linker structure comprising the above-mentioned bivalent alkylene group —R**—. A hydrogen bound directly to nitrogen may be replaced with a monovalent alkyl group selected amongst the same alkyl groups as R*— as long as they are not substantially counteracting the desired reactivity of the unsubstituted form of the nucleophilic centre. Thus the hydrogen in a thiol group and in a hydroxyl group cannot be replaced, for instance. Two replacing alkyl groups may form a cyclic structure together with atom to which they are attached, i.e. form a bivalent alkylene group e.g. selected amongst the alternatives for the —R**— group.

The bivalent structures —R**— and —R″— discussed above comprises next to the carrier a linker structure which does not negatively affect the desired effect of the nucleophilic centre of the scavenger structure. Such structures are not part of the invention and suitable such structures can be designed by the average-skilled person in the field.

In certain preferred scavenger structures there may be a second nucleophilic centre which
a) may be part of one of the organic groups, e.g. the R*— or the —R**— group, and b) contain a first heteroatom N, O or S (=Y¹) in the same manner as for the first nucleophilic centre. In principle this means that this second nucleophilic centre complies with the formula:

Y¹(—R″—)(—R′)ₘHₙ (III)

and the formula

—CH₂(Y⁴)_{o″}(C═Y³)_{n″}(Y²)_{m″} (IV)

where m, n, m″, n″, o″, Y¹, Y², Y³, Y⁴, —R″— and —R′ are selected in the same groups of variables as m, n, m′, n′, o′, X¹, X², X³, X⁴, —R″— and —R′of formula I and II. This includes that hydrogens (H) may be replaced as suggested for formulae I and II.

The heteroatom Y¹ preferably is part of
a) an —NH₂ group where the free valence preferably may bind to a sp³-hybridised carbon, Or
b) a thiol group —SH where the free valence preferably may bind to a sp³-hybridised carbon. Each of m″, n″ and o″ in formula IV is 0 in both (a) and (b).

The distance between the first heteroatom Y¹ and the first heteroatom X¹ is typically larger than two or three atoms with upper limits being e.g. 20 atoms with preference for 4, 5 or 6 atoms between these two heteroatoms. The distance should support intra-molecular cyclisation, typically via one or more addition reactions. This cyclisation typically comprises an addition reaction between the second nucleophilic centre and
a) a carbon-carbon or a carbon-heteroatom double bond formed as described above by reaction of the first nucleophilic centre with the starting aldehyde group, and/or
b) a reactive multiple C,C-bond present already in the starting aldehyde, such as a reactive double C,C-bond, e.g. α,β to the aldehyde group, and/or
c) a second keto or aldehyde carbonyl group provided such a group is present in the acetaldehyde molecule.

The result of the cyclisation is an n-membered ring-structure containing the first heteroatom Y¹ and the first heteroatom X¹ with n in n-membered being an integer ≥3 with preference for 5 or 6. Larger rings may also be formed, such as 7-, 8-, 9-, 10-, 11-, 12-, 13-, 14-membered rings, as long as steric considerations and relative positions of functional groups so admit. The cyclisation may be followed by rearrangement reactions, e.g. intramolecularly, and/or elimination reactions creating carbon-heteroatom double bond(s), ring-openings, etc.

### The Carrier

The selection of suitable carriers depends on the requirements of a particular use. The typical carrier is selected amongst macromolecular compounds, i.e. is a compound which has a molecular weight of ≥2000 dalton, preferably ≥10000 dalton or ≥50000 dalton, and preferably exhibits a polymeric structure, i.e. is a polymer which may be a homopolymer, copolymer or a chemical adduct between two or more polymers of different polymeric structure. Other suitable carriers may have molecular weights ≤2000 dalton and exhibit polymeric structure as indicated by the possibility of the low numbers of monomeric units discussed below, e.g. ≅20 and ≤100. The term “adduct polymer” in this context means a product formed by reacting two polymers exhibiting mutually reactive groups capable of forming covalent bonds that link the two polymers together upon reaction of the two mutually reactive groups with each other. See for instance WO 2009108100 (IPR-Systems AB) and references cited therein. Suitable macromolecular carriers may thus be selected amongst synthetic polymers (=man-made polymers), biopolymers (nature-made polymers such as polysaccharides, polypeptides, proteins, etc) and biosynthetic polymers where “biosynthetic polymer” refers to a macromolecular carrier or compound exhibiting both a synthetic polymeric structure and a biopolymeric structure. A carrier polymer may be cross-linked or not cross-linked. With respect to branching the polymer may be unbranched, i.e. linear, or branched including either hyperbranched or dendritic. The degree of branching may thus vary between 0 and 1, such as be ≥0.10 or ≥0.25≥0.5≥0.75 or ≥0.90 and/or ≤0.90 or ≤0.75 or ≤0.50 or ≤0.25 or ≤0.10. Cross-linked polymers are as a rule insoluble in aqueous liquids while the solubility of non-cross-linked polymers depend on the overall structure of the polymer, e.g. presence and amount of polar and/or hydrophilic groups. Carrier polymers may also be derivatized to contain non-polymeric or polymeric groups, for instance cross-links, substituents, charged or uncharged groups, scavenger structures (as discussed above), etc. Macromolecular carriers which are insoluble in aqueous liquids may have different physical and geometric shapes as discussed for support materials elsewhere in this specification.

The term polymer above includes organic as well as inorganic polymers.

The macromolecule or polymer used in the carrier may be water-insoluble and suspensible in aqueous liquid media (when in particle form).

Polymers and other macromolecules suitable as carrier material may be hydrophilic or hydrophobic with preference for hydrophilic. Pronounced hydrophobic macromolecular carriers are as a rule insoluble in aqueous liquids meaning that there may be a risk for host defence reactions with them and also that the availability of nucleophilic centres for reaction with bacteria may not be optimal. In order to overcome this kind of problems, it is often preferred to introduce hydrophilic groups on their surfaces (hydrophilization). The introduction of hydrophilic groups may among others be accomplished by
a) coating with a hydrophilic material,
b) selecting building blocks/monomers which exhibit hydrophilic groups and appropriate conditions during synthesis of the macromolecular compound, and
c) chemical derivatisation with hydrophilic groups subsequent to the synthesis of the basic hydrophobic polymer, etc.

The hydrophilicity of a group, structure or carrier molecule increases as a rule with an increase in the ratio r=the sum of the number of heteroatoms O, N and S divided by the sum of the number of carbon atoms. Hydrophilic groups/compounds typically have an r≥0.5, preferably ≥1.0, and for hydrophobic groups r<1.0, preferably ≤0.5. Typical hydrophilic groups are hydroxy, amino, amido, carboxy (including free acid carboxyl as well as carboxylate (ester ester and salt), etc. Typical hydrophobic groups are alkyls (CₙH₍₂ₙ₊₁₎—, CₙH₍₂ₙ₋₁₎—, CₙH₍₂ₙ₋₃₎—, etc), phenyls including alkyl phenyls, benzyl including

A carrier macromolecule typically comprises a polymer backbone which comprises ≥5, or more preferably ≥10 such as ≥25 different and/or identical monomeric units linked together. The polymer may carry projecting or pending polymeric and/or non-polymeric groups of various lengths and kinds. A carrier polymer is preferably hydrophilic with hydrophilic groups selected amongst those given elsewhere in this specification. The most preferred hydrophilic group is hydroxy with the preferred carrier polymers and/or other macromolecular carrier being selected by poly hydroxy polymers (PHP or PH-polymers) exhibiting ≅5, with preference for ≅10, such as ≅25 or ≅50 hydroxyl groups and/or ≅5 monomeric subunits each of which exhibits one, two, three, four or more hydroxyl groups per unit.

Typical polymers that may be present in polymeric carriers are a) polyester polymers, b) polyamide polymers, c) polyether polymers, d) polyvinyl polymers, e) polysaccharides, etc. A carrier may comprise one or more of these polymers/polymeric structures.

Polyester polymers are in particular obtained by polymerisation of a) monomers exhibiting at least one hydroxy group and at least one carboxy group, or b) a mixture containing monomers exhibiting two or more hydroxy groups and monomers exhibiting two or more carboxy group.

Polyamide polymers are in particular obtained by polymerisation of a) monomers exhibiting at least one amino group and at least one carboxy group, or b) a mixture containing monomers exhibiting two or more amino groups and monomers exhibiting two or more carboxy group. An important group of polyamides are those that exhibit polypeptide structure together with a plurality of hydroxy groups (PH-polymers). Suitable polyamide polymers of this kind are typically based on hydroxy-,amino-carboxylic acids as monomers, in particular with the amino group positioned a to the carboxylic group, e.g. serine, threonine, tyrosine, proline, etc.

Polyether polymers are typically used in combination with other polymeric structures, e.g. polymers of (a), (b), (d) and/or (e) above, which are polyfunctional with respect to the presence of groups such as hydroxy, amino, etc. Typical polyether polymers are polyethylene oxide and various copolymerisates between ethylene oxide and other lower alkylene oxides, lower epihalohydrins, etc.

Polyvinyl polymers which may be suitable as polymeric carriers in the invention are typically found amongst polymers containing one, two or more different monomeric units selected amongst hydroxyalkyl acrylates and methacrylates, N-hydroxyalkyl acryl- and N-hydroxyalkyl methacrylamides, hydroxyalkyl vinyl ethers, vinyl esters, etc. Polyvinyl alcohols are typically obtained by partial hydrolysis of polyvinyl esters meaning that polyvinyl alcohols that are preferred in the invention typically exhibit residual amounts of ester groups (≤10% or ≤5%).

Typical polysaccharides that may be present in carriers used in the invention include dextran, starch, agarose, agaropektin, cellulose, glucosamino glucanes (GAG), and derivates of these polysaccharides, etc. The most interesting polysaccharides are dextran, certain glucosamino glucanes (GAG) such as hyaluronic acid, etc.

A polymer to be used in the carrier may have been derivatized, e.g. cross-linked and/or functionalized after its synthesis.

The scavenger structure including the first, the optional second nucleophilic centre and the various heteroatoms discussed for the scavenger structures are typically part of one and the same organic group/substituent attached to the macromolecular carrier. In certain variants different parts of a scavenger structure may be part of different groups/substituents attached to the carrier and/or part of the carrier.

Sizes/molecular weights of suitable carrier polymers will among others depend on the actual application/use of the composition/method of the invention. Thus suitable polymeric carriers with respect to a particular polymeric structure and/or size may vary within a wide interval. Thus as a rule the number of monomeric subunits (mean value) of a polymer present in the carrier may be ≥20 or ≥100 or ≥200 or ≥300 or ≥500 or ≥1000 or ≥2000 or ≥20 000 or ≥50 000 and/or ≤50 000 or ≤20 000 or ≤2000 or ≤1000 or ≤500 or ≤300 or ≤200, or ≤100 (with the proviso that ≥-limit always is lower than a ≤-limit when these values are combined to define intervals). Preferred numbers of monomeric units may in some cases be found in the interval of 200-600 which in particular applies to the polyvinyl alcohol used in the experimental part.

Suitable numbers of scavenger structures or nucleophilic centres per monomeric unit of a polymer of the carrier will also depend on the use, the scavenger structure, bacteria, etc, and may thus be found within a wide interval, such as ≤80%, such as ≤50% or ≤20% with typical lower limits being 0.01% or 0.1% or 1% where 100% corresponds to one scavenger structure or nucleophilic centre per monomeric unit. For scavenger structures containing two or more nucleophilic centres the number of nucleophilic centres per monomeric unit may exceed 100%, such as ≥100% or ≥125% or ≥150%.

### Other Features of the Composition

AP is present in the composition as an AP-formulation in which AP is:
a) in dry form, for instance as free particles,
b) in dissolved form, typically in an aqueous liquid medium, and
c) in suspended/dispersed form, i.e. as water-insoluble particles suspended in an aqueous liquid medium,
d) attached to a support which is insoluble in aqueous liquid media.

The term “dissolved” in this context means that AP is present as a solute. AP particles comprise AP in a pure form or diluted with some solid material. Useful concentrations of AP in formulations according to (b) can be found within a broad interval. For liquid formulations e.g. within the interval of 0.1 μg-100 mg/mL.

The composition may in addition to AP contain buffers, salts, etc required for enabling acceptable conditions in vivo for the patient and for the reaction of AP with the bacteria. These constituents may be co-formulated with AP in the AP-formulation.

A potentially important variant of the inventive composition comprises formulations enabling production of cross-linked carrier polymers exhibiting a plurality of a nucleophilic structure that can be used as a scavenger structure (WO 2009108100, IPR-systems AB and references cited therein). The cross-linking may take place in vivo or ex vivo. In this variant the AP-formulation of the inventive composition is represented by at least two sub-formulations:
1) a first sub-formulation containing a macromolecular carrier exhibiting a plurality of a reactive nucleophilic group which has the potential of acting as a scavenger structure for bacterial growth or bacterial adhesion, and
2) a second sub-formulation containing a cross-linking reagent, preferably in the form of a polymer, and exhibiting (comprising) a plurality of a reactive electrophilic group which is capable of reacting in aqueous media with the reactive nucleophilic group of the macromolecular carrier to the introduction of covalent cross-links in the macromolecular carrier.

The macromolecular carrier in the first sub-formulation may be selected amongst the macromolecular carriers discussed above. When the cross-linking reagent in the second sub-formulation is a polymer this polymer may be selected amongst the polymeric macromolecular carriers discussed above.

The cross-linked carrier obtained by mixing the first sub-formulation with the second sub-formulation will contain a plurality of scavenger structures if the reactive nucleophilic group at the time of mixing is in molar excess compared to the reactive electrophilic group. Molar excess in this context typical means excess with a factor ≥2, such as ≥5 or ≥10 or ≥20. If the starting carrier and the cross-linking reagent is properly selected the obtained product will form a hydrogel in situ.

It can be envisaged that this kind of two-component compositions may be advantageous when administering by injection highly viscous solutions of high-molecular weight hyaluronic acid (desired polymer). Highly viscous solutions of hyaluronic acids are difficult to inject and administration of hyaluronic acid is often linked to a risk for adverse effects due to inflammatory-related reactions. These problems are likely to be reduced by injecting at the same location of a patient a composition comprising: p1 a) a first sub-formulation in which the macromolecular carrier is a low-molecular weight variant of hyaluronic acid in dissolved form (=a variant having a lower Mw than the Mw of the desired hyaluronic acid), and
b) a second sub-formulation in which the cross-linking reagent is also a low-molecular weight variant of hyaluronic acid in dissolved form,
   with the proviso that
i) that the reactive nucleophilic group in the first sub-formulation is in excess compared to reactive electrophilic groups in the second sub-formulation, and
ii) that the degree of substitution with respect to reactive electrophilic groups on the hyaluronic acid of the second sub-formulation (cross-linking reagent) is sufficiently low for producing a carrier in dissolved form, i.e. non-gel form, when the two sub-formulations are mixed with each other upon injection.

The mixing leads to a reaction between the reactive nucleophilic group and the reactive electrophilic group to the formation of a covalent cross-linking structure. Experimental testing is required for finding optimal degrees of substitution with respect to the reactive electrophilic group in the second sub-formulation relative to other reaction variables in order to obtain a carrier product in dissolved form, i.e. non-gel form.

The injection of the sub-formulations is preferably done in parallel, for instance with mixing of the formulations in the used syringe during or just before the injection is started. The techniques for this kind of injections and syringes to be used are well-known in the field; see for instance WO 2009108100 (IPR-Systems AB).

### Synthesis of the Active Principle

AP may be synthesized according to well-known protocols, for instance of the kinds given in WO 2009108100 (IPR-Systems AB) and references cited therein.

Water-Insoluble Support As mentioned above AP may be fixed to a water-insoluble support. This support material may be selected amongst support materials that have at least one or more of the following characteristics: a) in the form of particles, b) porous or non-porous particles or monoliths allowing or not allowing, respectively, aqueous liquids and/or bacteria to penetrate the support, c) rigid, d) soft, e) elastic, f) compressible, g) gellable (in particular to form a hydrogel when placed in contact with water), etc. The support may comprise plastics, glass, mineral, metal, etc. When the carrier of AP is insoluble in aqueous media the carrier as such may define its own solid support.

Supports may be designed as devices to be used in vivo and/or separate from an individual suffering from or being at risk for the inflammatory-related conditions to be treated or prevented. Typical devices are implants having AP exposed on their surface, e.g. stents, vascular prosthesis, nets, teeth, bones, joints, etc, patches, surgical dressings, plasters, filters, sutures, contrast media in the form of particles, etc. This also includes filter and adsorbent material for a) eliminating or reducing the growth and/or adhesion of bacteria brought in contact with animals including humans and/or b) ex vivo use, e.g. comprising eliminating or reducing the growth and/or adhesion of bacteria from biological fluids which derive from an individual and subsequently are to be returned to an individual suffering from or being at risk of bacteria or bacterial infections. Typical examples of biological fluids to be treated according (b) are blood, serum, plasma, etc.

Suitable supports typically comprise polymeric materials, e.g. comprising one or more polymer selected from the same polymers as the carrier polymers are selected. Typical carrier polymers are polysaccharides, e.g. cellulose, cross-linked dextran, agarose such as cross-linked agarose, etc, polyester polymers e.g. lactic acid copolymers such as polyglactin, polyethylenes, etc. Other kinds of support material may also be used, e.g. ceramic materials, plastics, mineral materials, metals, composite material, activated carbon, etc. Porous forms of these materials may be used as filters and/or adsorbent material.

Attachment to the support may be accomplished by mixing, coating, impregnating, etc the support with AP according to techniques known in the field. Alternatively, the macromolecular carrier of AP may be part of the material from which a support/device is made.

Contacting AP with Bacteria (Step (II) of the Method)

As discussed above the contacting of AP with the bacteria may take place in vivo of or separate from the individual to be treated.

The amount of AP in the composition which is brought into contact with the bacteria is effective in the sense that the bacteria-related response to be treated is mitigated and/or neutralised to an acceptable level. The suitable dosage (per administration) for in-vivo applications depends on the particular medical indication, formulation (e.g. kind of, support material, AP, scavenger structure, concentrations, etc), etc and thus is selected within a broad interval, e.g. 10⁻¹²-10² g with 10⁻⁶-10⁻³ g as a particularly interesting interval. Experimental testing is needed for individual cases.

Contacting in vivo comprises administration of the inventive composition systemically or locally depending on the particular medical indication treated and/or formulation used. Local administrations such as topical, dermal, nasal, intra-vitreal, intra-articular, oral, rectal, intra-osseous, etc are typically used when the condition to be treated is localised to the area of administration or at a location reachable for AP from this area. Systemic administrations such as parenteral administration, e.g. intravenous and subcutaneous administration or enteral administration, e.g. oral administration, etc, are mainly used when the conditions to be treated are occurring at locations not easily reachable by local administrations. Compare the discussion below on different medical indications.

### EXPERIMENTAL PART

### Synthesis Of Carbazate-Functionalized Polyvinyl Alcohol (PVAC)

Polyvinyl alcohol (5 g, 13-23 kDa) was dissolved in dimethyl sulfoxide (100 mL) while stirring for 1 hour at 80° C. under argon gas. Carbonyl diimidazole (10 g) was added and stirring continued at room temperature overnight. Hydrazine hydrate (10 mL) was then added, the reaction stirred overnight, and the product collected and purified by repeated precipitation in ethanol. The degree of substitution was determined spectrophotometrically by performing a trinitrobenzene sulfonic acid assay described elsewhere (Stephen L. Snyder and Philip Z. Sobocinski; Analytical Biochemistry 64, 284-288, 1975).

### EXAMPLE 1

### Antibacterial Effect of on 12 Different Bacteria Using Using PVAC

PVAC was dissolved in a physiological solution of sodium chloride, for assessment of its anti-bacterial effect in the following final concentrations: 5 mg/ml, 2.5 mg/ml, 1.25 mg/ml, 0.25 mg/ml, 0.05 mg/ml, 0.01 mg/ml, 2 ug/ml, 0.4 ug/ml and 80 ng/ml. The following bacteria were assessed:

The tested bacteria were Pseudomonas aeruginosa, Sphingomonas uppsaliensis, Klebsiella pneumonia, Klebsiella pneumoniae (ESBL—extended spectrum beta-lactamase), Staphylococcus epidermidis (KNS), Escherichia coli, Entreococcus faecium, Enterococcus faecalis, Enterobacter chloacae, Streptococcus pyogenes, Staphylococcus aureus, and Bacillus cereus.

In 96-well plates, PVAC was mixed with either 10³ or 10⁵ bacteria from each of the species outlined above, with the addition of bacterial nutrient growth medium providing final PVAC concentrations as specified above. Bacterial growth was assessed over time using spectrophotometry, determining the optical density (OD) of the solution. Increased bacterial growth leads to an increase in OD. The anti-bacterial effect was further confirmed by sampling bacteria from the 96-well plates at various time points and growing them on agar plates.

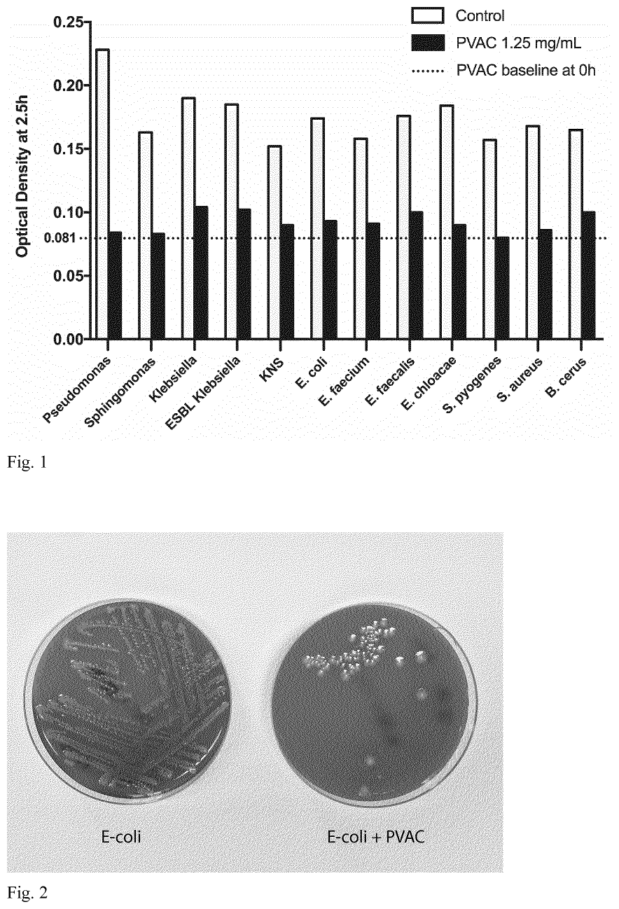

PVAC displayed a potent anti-bacterial effect on all assessed bacterial species, including multi-resistant ESBL bacteria, with OD near the baseline at 2.5 h (FIG. 1), whereas bacterial growth in the non-PVAC-containing controls were exponentially higher.

The effect seen on OD was confirmed using growth on agar plates, with a potent anti-bacterial effect evident by a reduction in the number of colony forming units for E. coli (FIG. 2).

While the invention has been described and pointed out with reference to operative embodiments thereof, it will be understood by those skilled in the art that various changes, modifications, substitutions and omissions can be made without departing from the spirit of the invention.

1. An active principle for use in prevention or treatment of bacteria-induced infection and disease, wherein the active principle comprises a carrier which exhibits a plurality of a scavenger structure,
said scavenger structure comprising a nucleophilic centre complying with the formula
X¹(—R″—)(—R′)ₘHₙ (I)
wherein
a) X¹ is a single-bonded heteroatom selected amongst N, O and S and exhibits a free electron pair,
b) m is 0 or 1 and n is 1 or 2 with the sum of m plus n being 2 for X¹=N and 1 for X¹=S and O,
c) —R″— is a bivalent organic group providing attachment to the carrier via one of its free valences and direct attachment to the heteroatom X¹ at the other one of its free valences, and
d) R′— is a monovalent organic group directly attached to the heteroatom X′ via its free valence.
wherein the bacteria-induced infection and disease are selected from infection and
disease in connection with mouth, dental or throat infections, systemic infection,
alone or as an adjuvant treatment to antibiotics, infections in skin sores or ulcers, eye
infections, ear infections, cystitis in individuals with catheter, venereal diseases in
individuals, bacterial growth in bags containing blood or other blood components,
including plasma and thrombocytes, respiratory infections, infections deep in body
cavities or abscesses, central nervous infections, or prevention of
bacterial infections in immunosuppressed patients.

2. The active principle for use according to claim 1, wherein that either one or both of the organic groups R′— and —R″—, with preference for —R″—, comprise a structure of the formula:
—CH₂(X⁴)_{o′}(C═X³) (II)
wherein
a) each of m′, n′ and o′ is 0 or 1, with preference for m′ being 1 with further preference for either one or both of n′ and o′ also being 1,
b) each of X², X³, and X⁴, is selected amongst NH and a heteroatom S or O, with preference for either one or both of X² and X⁴ being selected amongst NH and O with further preference for X³ being selected amongst NH, O and S,
c) the left free valence provides binding to a monovalent alkyl group R*— or to the carrier via at least a bivalent alkylene group —R**—, each of which two groups comprises the methylene group —CH₂— shown in formula II, and
d) the right free valence binds directly to the first heteroatom X¹.

3. The active principle for use according to claim 1, wherein the nucleophilic center is part of a group selected amongst
a) amino groups,
b) hydrazide groups a semicarbazide group, a carbazate group, a thiosemicarbazide group, a thiocarbazate group,
c) aminooxy groups, and
d) thiol groups.

4. The active principle for use according to claim 1, wherein the carrier is a macromolecular carrier and/or is water-soluble or water-insoluble.

5. The active principle for use according to claim 1, wherein a) the carrier is water-insoluble and defines a support, and/or b) the active principle is attached to a water-insoluble support.

6. The active principle for use according to claim 1, wherein the scavenger structure is capable of undergoing an addition reaction with a carbonyl group of an aldehyde group and/or with a reactive carbon-carbon multiple bond to which is directly attached a carbonyl group.

7. The active principle for use according to claim 1, wherein the active principle is carbazate-functionalized polyvinyl alcohol.

8. A non-medical antibacterial composition comprising an active principle and a suitable carrier and/or an adjuvant, wherein the active principle comprises a carrier which exhibits a plurality of a scavenger structure, said scavenger structure comprising a nucleophilic centre complying with the formula
X¹(—R″—)(—R′)ₘHₙ (I)
wherein
a) X¹ is a single-bonded heteroatom selected amongst N, O and S and exhibits a free electron pair,
b) m is 0 or 1 and n is 1 or 2 with the sum of m plus n being 2 for X¹=N and 1 for X′ =S and O,
c) —R″— is a bivalent organic group providing attachment to the carrier via one of its free valences and direct attachment to the heteroatom X¹ at the other one of its free valences, and
d) R′— is a monovalent organic group directly attached to the heteroatom X¹ via its free valence.

9. A mouthwash or gargling solution for reducing the bacterial content in the oral cavity to improve the smell of the breath, comprising an active principle and a suitable carrier and/or an adjuvant, wherein the active principle comprises a carrier which exhibits a plurality of a scavenger structure, said scavenger structure comprising a nucleophilic centre complying with the formula
X¹(—R″—)(—R′)ₘHₙ (I)
wherein
a) X¹ is a single-bonded heteroatom selected amongst N, O and S and exhibits a free electron pair,
b) m is 0 or 1 and n is 1 or 2 with the sum of m plus n being 2 for X¹=N and 1 for X¹=S and O,
c) —R″— is a bivalent organic group providing attachment to the carrier via one of its free valences and direct attachment to the heteroatom X¹ at the other one of its free valences, and
d) R′— is a monovalent organic group directly attached to the heteroatom X¹ via its free valence.

10. An antibacterial sanitizing solution comprising an active principle and a suitable carrier and/or adjuvant for pre- or perioperative sanitizing of medical implants during surgery, wherein the active principle comprises a carrier which exhibits a plurality of a scavenger structure,
said scavenger structure comprising a nucleophilic centre complying with the formula
X¹(—R″—)(—R′)ₘHₙ (I)
wherein
a) X¹ is a single-bonded heteroatom selected amongst N, O and S and exhibits a free electron pair,
b) m is 0 or 1 and n is 1 or 2 with the sum of m plus n being 2 for X¹=N and 1 for X¹=S and O,
c) —R″— is a bivalent organic group providing attachment to the carrier via one of its free valences and direct attachment to the heteroatom X′ at the other one of its free valences, and
d) R′— is a monovalent organic group directly attached to the heteroatom X¹ via its free valence.

11. Pharmaceutical composition comprising the active principle defined in claim 1 and pharmaceutically acceptable adjuvants for use in the prevention or treatment of bacteria-induced infection and disease in connection with mouth, dental or throat infections, systemic infection, alone or as an adjuvant treatment to antibiotics, infections in skin sores or ulcers, eye infections, ear infections, cystitis in individuals with catheter, venereal diseases in individuals, bacterial growth in bags containing blood or other blood components, including plasma and thrombocytes, respiratory infections, infections deep in body cavities or abscesses, central nervous infections, or prevention of bacterial infections in immunosuppressed patients.

12. A surface coating comprising an active principle, wherein the active principle comprises a carrier which exhibits a plurality of a scavenger structure, said scavenger structure comprising a nucleophilic centre complying with the formula
X¹(—R″—)(—R′)ₘHₙ (I)
wherein
a) X¹ is a single-bonded heteroatom selected amongst N, O and S and exhibits a free electron pair,
b) m is 0 or 1 and n is 1 or 2 with the sum of m plus n being 2 for X¹=N and 1 for X¹=S and O,
c) —R″— is a bivalent organic group providing attachment to the carrier via one of its free valences and direct attachment to the heteroatom X¹ at the other one of its free valences, and
d) R′— is a monovalent organic group directly attached to the heteroatom X¹ via its free valence, or comprises the non-medical antibacterial composition according to claim 8.

13. An object having an inner and an outer surface wherein at least part of the inner surface and/or outer surface exhibit an antibacterial surface coating as defined in claim 12.

14. The object according to claim 13, wherein the object is a blood bag.

15. The object according to claim 13, wherein the object is a catheter.

16. The active principle for use according to claim 1, wherein the carrier is a macromolecular carrier and/or is water-soluble or water-insoluble and exhibits polymer structure.

17. The active principle for use according to claim 2, wherein the nucleophilic center is part of a group selected amongst
a) amino groups,
b) hydrazide groups, a semicarbazide group, a carbazate group, a thiosemicarbazide group, a thiocarbazate group,
c) aminooxy groups, and
d) thiol groups.

18. The active principle for use according to claim 2, wherein the carrier is a macromolecular carrier and/or is water-soluble or water-insoluble.

19. The active principle for use according to claim 3, wherein the carrier is a macromolecular carrier and/or is water-soluble or water-insoluble.

20. The active principle for use according to claim 2, wherein a) the carrier is water-insoluble and defines a support, and/or b) the active principle is attached to a water-insoluble support.
